(19) United States
(12) Patent Application Publication
(10) Pub. No.: US 2024/0404656 A1
(43) Pub. Date: Dec. 5, 2024

(54) A COMPUTER IMPLEMENTED METHOD FOR ASSESSING AND DETERMINING A COMPLEXITY LEVEL OF A CLINICAL TRIAL STUDY

(71) Applicant: NOVARTIS AG, Basel (CH)

(72) Inventors: Naomi EBHOMIELEN, Dublin (IE); Florian BIEBER, Darmstadt, Hessen (DE); Stephen Sung Yong CHO, Lexington, MA (US); Andrew McQuoid KNUEVEN, Medford, MA (US); Wolgfang Christian KOTHNY, Lorrach (DE); Lidija KOVACIC, Widen, Aargau (CH); Ashwini MATHUR, Shankill, Dublin (IE); Suzanne MCCREDDIN, Whitehall, Dublin (IE); Sarah WHELAN, Crumlin, Dublin (IE)

(21) Appl. No.: 18/694,271

(22) PCT Filed: Sep. 21, 2022
(86) PCT No.: PCT/IB2022/058938

§ 371 Date: Mar. 21, 2024

Related U.S. Application Data
(60) Provisional application No. 63/247,602, filed on Sep. 23, 2021.

(51) Int. Cl. G16H 10/20

(52) U.S. Cl. CPC G16H 10/20

(57) ABSTRACT

The present invention relates to a computer-implemented method for assessing and determining a complexity level of at least a portion, or pillar, of a clinical trial study. The method may be implemented by a computing device including at least a processor; a memory and an input and/or output elements. It comprises the steps of selecting and/or defining a clinical trial protocol and, based on the selection and/or definition thereof, activating and/or generating data entries for a set of corresponding parameters correlated to a set-up of the portion of the clinical trial study. The parameters refer to objectives; and/or design; and/or methods; and/or patient assessment procedures and/or patient data collection schedules of the clinical trial study. The method further comprises the steps of automatically assigning a score value to each parameter in the set of parameters; applying statistical rules to the score value of each parameter to obtain a scaled score value; and calculating a complexity level for the portion of clinical trial study, based on the scaled score values of each parameter in the set of parameters.

### TECHNICAL FIELD

The present invention relates to a computer-implemented method for estimating a complexity level of clinical trial study. In particular, the computer-implemented method according to the present invention assesses and outputs a complexity level of at least a portion, or pillar, of a clinical trial study, whereby the method can calculate an overall complexity level score for the entire clinical trial study, while also providing a breakdown complexity level score for portions, or pillars, thereof.

The present invention also relates to a computing system, designed to carry out the method, wherein the computing system comprises a computing device including one or more processors; one or more input and/or output elements; a memory; and one or more programs stored in the memory including instructions for implementing the method.

The present invention further relates to a non-transitory computer-readable storage medium storing one or more programs configured to be executed by one or more processors of an electronic device with a memory and one or more input and/or output elements, the one or more programs including instructions for carrying out the above-mentioned computer-implemented method.

The computer-implemented method according to the present invention can be run on traditional computers, as well as on mobile computing platforms, in order to identify key parameters driving the complexity level of a clinical trial study. Such method can be implemented in computer programs executing on digital mobile application modules or App modules.

The computer-implemented method according to the present invention can therefore also be advantageously employed in the context of designing clinical trials.

### BACKGROUND ART

Normally, each clinical trial study follows an expressly developed clinical protocol, establishing how the clinical trial will be conducted. The protocol includes instructions for setting objective(s) of the clinical trial project and for deciding on methodologies to be employed, on statistical considerations to be used and on organizational procedures to be carried out, also to ensure safety of the subjects involved and integrity of the data collected according to Good Clinical Practice guidelines. GCP guidelines comprise standards on how clinical trials should be conducted and define the roles and responsibilities of institutional review boards, clinical research investigators, clinical trial sponsors, and monitors.

Several factors impact clinical trial costs, inter alia, study size or the number of patients to be recruited; locations, as in number of countries and number of clinical sites; therapeutic area; drug type; and the specific tests and procedures needed per protocol. The study size is in turn closely related to the study phase. International phase 3 studies may involve considerably more patients than phase 1 trials.

Given the multibranched nature of clinical trials, clinical trial planning and specifically cost calculation can be arduous and time consuming. Despite the importance of knowing the clinical trial bill, biotechnology and pharmaceutical companies struggle to get a clear picture of all the costs involved in a clinical study through the stages of drug development.

Moreover, awareness of costs correlated to conducting clinical trials is just one aspect of structuring and planning clinical trials within drug development programs.

In fact, apart from funding, it would be ideal to provide a quantifiable, repeatable measure of the various efforts required to conduct clinical trial studies, which enables biotechnology and pharmaceutical companies to effectively build a strategy for carrying out and optimizing the set-up of clinical trial studies.

Currently there are no tools on the market which comprehensively illustrate the impact of each of the several fundamental variables involved in a given clinical trial study on the overall complexity of such study.

In practice, publicly available tools for structuring and planning clinical trials are typically simple Excel-based spreadsheets, oftentimes developed based on just very partial budget reasonings. Also, these tools are usually locally developed, in a way that is limited to a specific study, implying labor-intensive and sometimes arbitrary validation of individual items when adjusting to different clinical trial studies. At the moment, none of these tools allows to precisely account for each of the complexity drivers of a clinical study. Consequently, planning cost structures and allocation of resources for clinical trial studies remains an empirical process.

Therefore, there is a need for a computer-implemented method to provide a consistently repeatable, reliable, precise and fast estimation of the overall complexity of clinical studies, preferably enabling an automatic determination of complexity measures or metrics for each of the several, distinct activities and characteristics associated thereto, thus allowing to assess and optimize clinical trials' protocols.

### DISCLOSURE OF THE INVENTION

According to the invention, this need is settled by a computer-implemented method for assessing and determining a complexity level of at least a portion, or pillar, of a clinical trial study as it is defined by the features of independent claim 1; by a system as it is defined by the features of independent claim 18; and by a non-transitory computer-readable storage medium as it is defined by the features of independent claim 19. Further embodiments are herein described, for example, in the dependent claims.

In particular, the invention deals with a computer-implemented method for identifying parameters, or factors, which drive the complexity within a given clinical trial study and for estimating how each of these parameters, or factors, contributes to an overall complexity level of the clinical trial study, or at least to a portion, or pillar, of the clinical trial study. At the same time, the present computer-implemented method allows a user to compare the given clinical trial study against an updated, growing benchmark of clinical trial studies, at an overall complexity level corresponding to the whole clinical trial study or to a portion thereof, and/or at an individual parameter level. The method can be implemented on a computing device including one or more processors; a memory and/or storage; and one or more input and/or output elements.

Thus, the method according to the present invention provides a platform to plan, track, simulate and share, or propose, outcomes of a clinical trial study. The clinical trial study, or a portion thereof, is set up into a series of activities. To each one of these activities a respective parameter is correlated, each parameter being characterized by a score value which is a mathematical expression of the complexity or effort required to carry out the specific activity. Accordingly, the method helps to gain awareness on the impact of each of these activities on the complexity of the clinical trial study, based on the score values attributed to the parameters correlated to the activities, allowing to proactively shape and modify the layout of the clinical trial study in a consequent way.

The computer-implemented method of the present invention allows to assess and determine a complexity level of at least a portion, or pillar, of a clinical trial study. It comprises a first step of selecting and/or defining a clinical trial protocol, by an input element of a computing device which includes at least a processor; a memory and an input and/or output elements. The selection and/or definition of the protocol can be manual, executed by an operator/user via the input element, and/or automatic, e.g. by automatic auto-population.

Based on the above-mentioned protocol selection and/or definition, the method comprises a step of activating and/or generating data entries for a set of corresponding parameters correlated to a set-up of the portion of the clinical trial study. These parameters capture factors driving the complexity level of the portion of the clinical trial study and refer in general to activities encompassed by the clinical trial study (or by the specific portion of the clinical trial study taken into analysed), such as objectives; and/or design; and/or methods; and/or patient assessment procedures and/or patient data collection schedules of the clinical trial study.

A protocol defines how a clinical trial study will be conducted, including objective(s), design, methodology, statistical considerations and organization thereof; and it ensures the safety of the trial subjects and integrity of the data collected.

The computing device's processor automatically assigns a score value to each parameter in the set of parameters. As mentioned, the score value is a mathematical expression of the complexity incurred in carrying out a specific activity associated with the parameter, and of the effort required for completing it.

Further to that, the method comprises a step of applying statistical rules to the score value of each parameter, or to a distribution of such score value, to obtain a scaled score value for the parameter. Based on the application of such statistical rules, it is substantially calculated how each parameter's score value compares to an average score value of the same parameter. The average score value for each parameter can be derived based on benchmark score values of retrospective/previously assessed clinical trial studies for the same parameter.

Finally, the method of the present invention then comprises a step of calculating a complexity level for the portion of clinical trial study which is being analysed, based on the score values of each parameter in the set of parameters. For this purpose, advantageously the score value of each parameter is adaptively based on the benchmark score values of the retrospective/previously assessed clinical trial studies, for instance the same benchmark score values used for deriving the average score value of each parameter, as above mentioned.

Accordingly, a ‘live’ benchmark based on a continuously built and updated database of clinical trial studies is preferably used to establish a realistic score value of each parameter in the set of parameters, and consequently to calculate an average score value for each parameter with reference to a representative benchmark. Such a “live” or “growing” benchmark can include all kinds of clinical trials, or be limited to clinical trial studies in a specific disease area. Employing a “live” benchmark, progressively updated with data of newly implemented clinical trial studies, as opposed to establishing a set static threshold for the complexity level, allows the method according to the present invention to better capture the evolving status and reality of clinical trial studies as they are being carried out.

In some embodiments, for each parameter, a score value from a scoring table is applied. In some embodiments, the score value from the scoring table is within a range of values comprised between 0-200; the median value for each scored parameter can be set at 100. Each parameter's score value stored in a scoring table, reflects at any rate the relative importance of the parameter score value as gathered from the abovementioned updated benchmark, in way as to prevent excessive weight for extreme values. The parameter score value substantially represents a “work effort unit” can be based on at least one of procedure type, procedure cost, procedure time, and procedure phase. The value of parameter is then assigned based on case specific input data, such as the number of occurrences a given procedure is repeated in compliance with the protocol, and on the scoring table.

In some embodiments, the clinical trial study comprises a multiplicity of portions, or pillars. For each portion, or pillar, of the clinical trial study, a pillar complexity score value is calculated by averaging the scaled score value of each parameter comprised in each of portion, or pillar

In some embodiments, the clinical trial study comprises at least one, or a combination, of the following portions, or pillars: a Clinical Trial Design pillar; a Patient & Site Burden pillar; an Operational Burden pillar. In some of these embodiments, the Clinical Trial Design pillar can comprise fourteen parameters; the Patient & Site Burden pillar can comprise seven parameters; and the Operational Burden pillar can comprise eight parameters; the clinical trial study thus comprising overall twenty-nine parameters.

By way of example, the Clinical Trial Design pillar can comprise the parameters: Primary Objectives; Secondary Objectives; Exploratory Objectives; Study Arms; Washout Period (Yes/No); Allocation; Intervention Model; Stratification (Yes/No); Masking; Trial Blinding Schema; No. of inclusion/exclusion criteria; Route of Study Drug; Comparison; Number of sub studies.

Moreover, by way of example, the Patient & Site Burden pillar can comprise, in a first Part A of the assessment, the parameters: Study Duration (Weeks); Total Number of Planned Visits; %, or percentage, of Homebased Visits; Age Group; Overnight stay required for study procedures?; %, or percentage, of visits with a blood draw. In addition to that, the Patient & Site Burden can comprise a distinct Patient & Site Burden Assessment Part B parameter, which will be in the following elucidated in connection with an adjustment factor, or weight, to be applied to the score value of each parameter, in order to obtain an adjusted score value “x” as in the below standardisation formula.

Further, by way of example, the Operational Burden pillar can comprise the following parameters: countries; sites; expected screen fail rates; total patients; roll-over patient design; adjudication; interim analysis; vendors.

In some embodiments, an overall complexity level score value for the clinical trial study is calculated by averaging the scaled score values determined for each of the portions, or pillars.

In some embodiments, the scaled score value of each parameter is the standardized score value, also designatable by SV, for each parameter. Accordingly, a pillar standardized complexity score value can be calculated by averaging the standardized score value of each parameter comprised in each portion, or pillar. Analogously, an overall standardized complexity level score value for the clinical trial study can be calculated by averaging the standardized score values determined for each of the portions, or pillars.

Standardized complexity is thus advantageously adaptive per parameter and reflects the current status of the benchmark.

In the embodiments wherein the scaled score value of each parameter is the standardized score value (SV) for each parameter, the standardized score value (SV) can be calculated according to the following formula:

$$SV = ( x - mean ) / standard deviation$$

wherein:
“x” is an adjusted score value for each parameter, derived from the above mentioned predefined scoring table reflecting the relative importance of the score value of the parameters;
“mean” is the mean of each score value, based on the benchmark score values of the retrospective/previously assessed clinical trial studies for the same parameter; and
“standard deviation” is a value calculated as the square root of variance, by determining each parameter's score value deviation relative to the mean.

Such standardized function is also adjusted to ensure not one parameter overwhelms the final scoring and allows for differences to be detected more easily than, e.g., through use of normalisation.

In some embodiments, the scoring table follows a sigmoidal distribution curve for each parameter.

The “mean” value reflects how each parameter's score value compares to an average score value of the same parameter. In the above formula, the “standard deviation” ultimately measures the amount of variation, or dispersion, of a distribution of the score values of a parameter in the set, that is how many standard deviations the parameter's adjusted score value score “x” is below or above the benchmark.

A scaling such as a standardization as above defined advantageously makes sure that no single parameter takes over the whole model.

In some embodiments, the “mean” of each parameter's score value, used in the above formula, equals to the 90% Trim Mean, that is the mean of the middle 90% benchmark score values for the same parameter, as derived from the benchmark database comprising values of retrospective/previously assessed clinical trial studies. A TRIMMEAN function takes the value data set from the benchmark values for a given parameter; orders the data from smallest to largest value; eliminates the bottom 5% and the top 5% values, leaving just the middle 90% values, and finally calculates the average value of these remaining 90% values. Cutting off the 5% ‘extreme’ score values at each end, or tail, of the distribution curve for each parameter is advantageous, because it eliminates outliers.

In some embodiments, an adjustment factor, or weight, is employed for the score value of each parameter, to obtain an adjusted score value. This adjustment factor, or weight is preferably applied prior to the calculation of the standardized score value (SV) introduced above. The adjustment factor, or weight is especially configured to prevent that a score value of any parameter, such as those included in the Patient & Site Burden pillar, dominates over the score value of other parameters.

In fact, in some embodiments, the adjustment factor, or weight, is calculated just based on the Patient & Site Burden pillar score value, to prevent that the Patient & Site Burden pillar score value dominates over score values of other pillars' parameters.

In some embodiments, the adjustment factor, or weight, can be calculated in the Part B Assessment, based on the relative complexity score value of the Patient & Site Burden pillar, as compared to the median of all clinical trial studies comprised in the benchmark of retrospective/previously assessed clinical trial studies. In some embodiments, the adjustment factor, or weight, is calculated as the average complexity score value of the Patient & Site Burden pillar, calculated across all clinical trial studies comprised in the benchmark of retrospective/previously assessed clinical trial studies, divided by an estimated median of each scored parameter, such as 100. In these instances, the complexity score value of the Patient & Site Burden pillar can be the average of the sums of all scores multiplied by the number of occurrences, wherein a standardization function has not yet been applied.

In some embodiments, each parameter comprised in the Patient & Site Burden pillar is assigned a score value from a distinct, pre-defined scoring table across all clinical trials to date, in a so-called Patient & Site Burden Assessment Part B table, having values separately dedicated to each of the Patient Burden & Site Burden parameters. Therefore, two actual scores can be used for each distinct item or element or activity: one is related to Patient Burden portion, measuring invasiveness of assessment to the patient from a patient point of view; and the other one is related to Site Burden portion, measuring how difficult it is to operationalise the assessment by the investigative site from a sponsor point of view. They are not necessarily equal for each Patient or Site Burden item.

Each score value, respectively for the Patient portion and for the Site portion of the Patient & Site Burden pillar, is to be multiplied by the number of occurrences, or repetitions or reiterations, needed in compliance with the clinical trial protocol of the current study and subsequently summed. The clinical trial study being assessed is assigned the complexity score value for the Patient & Site Burden pillar as sum of the two Patient and Site sums thus obtained. This part of the scoring may be not capped and the score can reach much higher values than for other parameter scores, depending on the trial protocol including duration of the study and the frequency of each intervention. The Patient & Site Burden score from the above distinct, pre-defined scoring table across all clinical trials to date (i.e. Part B Assessment) is one score transferred to the Part A Assessment. In Part A Assessment, there are a few other scored elements (e.g. Study Duration (Weeks); Total Number of Planned Visits; % Homebased Visit; Age Group; Overnight stay required for study procedures?; % of visits with a blood draw) composing the overall Patient and Site Burden category: scored as per the original explanation above.

By way of example, each Patient & Site Burden element, or item or activity, can score 0-5 based on how invasive it is for the patient, when linked to the Patient Burden portion; and/or how difficult to operationalize for the site, when linked to the Site Burden portion.

Moreover, after summing up each value item in the Patient & Site Burden Part B Assessment table as above described, the average for a Patient & Site Burden Part B Assessment parameter, across all studies in the benchmark, can be calculated as a measure of relative complexity.

Further to that, starting from the assumption that each scored parameter has median of 100, the uncapped total score obtained from such Part B Assessment table across all trials to-date can be divided by 100 and then used as the actual weight as above introduced. By way of example, if the average of the Patient & Site Burden Part B Assessment is 220 across all previous studies, the relative weight of 2.2 can be applied to all other scored parameters for the study being evaluated-except naturally for the distinct Patient & Site Burden Part B Assessment parameter itself-prior to the standardization.

In short, relative to the example given above wherein the clinical trial study comprises overall 29 parameters, the original score value for each of the 29 parameters, except for the distinct Patient & Site Burden Part B Assessment parameter, can be multiplied by such weight of 2.2 obtained from the Part B Assessment.

In some embodiments, when selecting and/or defining a clinical trial protocol, the computing device automatically presents an initial user interface to an operator, comprising a preliminary multiplicity of clinical trial protocol data entries activated and/or generated based on an initial choice by an operator. For instance, the operator's initial input can be one of a category of therapeutic area and/or of a developmental unit. Subsequently the computing device can present to the operator clinical trial protocol data entries adaptively adjusted, for instance based on a probability value that the set of corresponding parameters drives the complexity level of the portion of the clinical trial study currently analysed. Thanks to that, the operator, or user, is continuously and dynamically guided in the assessment and/or in the identification of factors contributing to the complexity of the clinical trial study. Consequently, the operator is enabled to at least partially re-shape the current clinical trial study is a way that he may deem appropriate. Thus, the method according to the present invention actually becomes an interactive tool which allows to assess, plan and optimize a clinical trial study at several levels, from the overarching scheme down to the single constituent elements or activities, while confronting it to a dynamic, ever updated benchmark of clinical trial studies.

In some embodiments, a graphical user interface displayed on a display screen of the computing device is configured to visually convey to a user, or operator, a measure of the impact on the complexity level of each portion, or pillar, and/or of each parameter comprised in the clinical trial study, as a result of a current selection and/or definition of a clinical trial protocol.

In some embodiments, the graphical user interface can be interactive, in that it changes in real-time, based on modified selection and/or definition of a clinical trial protocol by a user, or operator.

In some embodiments, the graphical user interface allows to visualize the complexity level of the clinical trial study, as an overall standardized score value. Analogously, the complexity level of a portion, or pillar, of the clinical trial study can be visualized. The information thus displayed can be put in relation to other clinical trial studies comprised in the benchmark of retrospective/previously assessed clinical trial studies, for a quick reference which can be an immediate aid in an operator's decision making and enhance the perception of the assessment's findings. The graphical user interface can allow to visualize the complexity level of the clinical trial study, either within clinical trial studies belonging to a specific therapeutic area or developmental unit; or across all clinical trial studies; or filtering by clinical trial study phase.

In some embodiments, the benchmark score values of the retrospective/previously assessed clinical trial studies are updated and stored on the memory, prior to applying statistical rules to the score value of each parameter for assessing and determining the complexity level of the portion, or pillar, of the clinical trial study. Thus, the latest status of the benchmark is ensured.

The present invention also refers to a system comprising a computing device including one or more processors; one or more input and/or output elements; memory; and one or more programs stored in the memory including instructions to execute the method above described. In order to allow more flexibility from remote and support planning of clinical trials even from remote or mobile computing devices; also a hand-held or wearable computing devices can be used, for instance a tablet, watch or a smart-phone.

The present invention also refers to a non-transitory computer-readable storage medium storing one or more programs configured to be executed by one or more processors of an electronic device with one or more input and/or output elements and a memory, wherein the one or more programs are designed to include instructions to execute the method above described.

### BRIEF DESCRIPTION OF THE DRAWINGS

The method and the system and according to the invention are described in more detail herein below by way of exemplary embodiments and with reference to the attached drawings, in which:

FIG. 1 illustrates a view of an exemplary graphical user interface on a computing device implementing the method according to the present invention, the view prompting an operator to input descriptive features in generated data entries correlated to a set-up of a clinical trial study in compliance with a clinical trial protocol;

FIG. 2 illustrates an example of parameters referring to objectives; design; methods; patient assessment procedures and patient data collection modes in a Trial Design pillar, or portion, of a clinical trial study, wherein the method according to the present invention calculates a score for each parameter, adaptively based on benchmark score values of retrospective/previously assessed clinical trial studies for the same parameter; as well as a standardized score value of each parameter relative to the benchmark, representing a standardized complexity;

FIGS. 3A and 3B illustrate examples of parameters referring to objectives; design; methods; patient assessment procedures and patient data collection modes in a Patient and Site Burden pillar, or portion, of a clinical trial study, wherein the method according to the present invention calculates a score for each parameter, adaptively based on benchmark score values of retrospective/previously assessed clinical trial studies for the same parameter; as well as a standardized score value of each parameter relative to the benchmark, representing a standardized complexity;

FIGS. 4 and 5 illustrate exemplary sequences of score values for two selections of parameters, both for the Patient portion and for the Site portion of the Patient & Site Burden pillar, in compliance with the clinical trial protocol of a given study, subsequently summed to obtain a combined score value for each parameter within the Patient and Site Burden pillar, wherein the example of FIG. 4 comprises a sequence of parameters for medical procedures with a high complexity level, whereas the example of FIG. 5 comprises a sequence of parameters for medical procedures, tests and methods with a low complexity level;

FIG. 6 illustrates an example of parameters referring to objectives; design and patient data collection modes in an Operational Burden pillar, or portion, of a clinical trial study, wherein the method according to the present invention calculates a score for each parameter, adaptively based on benchmark score values of retrospective/previously assessed clinical trial studies for the same parameter; as well as a standardized score value of each parameter relative to the benchmark, representing a standardized complexity;

FIGS. 7A and 7B collectively show a graphical user interface wherein FIG. 7A shows plots on a graph and FIG. 7B the legends associated with the plots. FIG. 7 are configured to visually convey to a user, or operator, a measure of the complexity level of several clinical trial studies in relation to an updated benchmark, across clinical trial studies carried out in a selectable array of developmental units; the graphical user interface allowing an operator to visualize the complexity level of studies belonging to a specific developmental unit; or across all clinical trial studies; and/or filtering by clinical trial study phase; and

FIG. 8 is a diagrammatic illustration of an overall process flow according to exemplary embodiments of the present invention.

### DESCRIPTION OF EMBODIMENTS

In the following description certain terms are used for reasons of convenience and are not intended to limit the invention. The terms “right”, “left”, “up”, “down”, “under” and “above” refer to directions in the figures. The terminology comprises the explicitly mentioned terms as well as their derivations and terms with a similar meaning. Also, spatially relative terms, such as “beneath”, “below”, “lower”, “above”, “upper”, “proximal”, “distal”, and the like, may be used to describe one element's or feature's relationship to another element or feature as illustrated in the figures. These spatially relative terms are intended to encompass different positions and orientations of the devices in use or operation in addition to the position and orientation shown in the figures. For example, if a device in the figures is turned over, elements described as “below” or “beneath” other elements or features would then be “above” or “over” the other elements or features. Thus, the exemplary term “below” can encompass both positions and orientations of above and below. The devices may be otherwise oriented (rotated 90 degrees or at other orientations), and the spatially relative descriptors used herein interpreted accordingly. Likewise, descriptions of movement along and around various axes include various special device positions and orientations.

To avoid repetition in the figures and the descriptions of the various aspects and illustrative embodiments, it should be understood that many features are common to many aspects and embodiments. Omission of an aspect from a description or figure does not imply that the aspect is missing from embodiments that incorporate that aspect. Instead, the aspect may have been omitted for clarity and to avoid prolix description. In this context, the following applies to the rest of this description: If, in order to clarify the drawings, a figure contains reference signs which are not explained in the directly associated part of the description, then it is referred to previous or following description sections. Further, for reason of lucidity, if in a drawing not all features of a part are provided with reference signs it is referred to other drawings showing the same part. Like numbers in two or more figures represent the same or similar elements.

The following description sets forth exemplary systems, devices, methods, parameters, and the like. It should be recognized, however, that such description is not intended as a limitation on the scope of the present disclosure but is instead provided as a description of exemplary embodiments. For example, reference is made to the accompanying drawings in which it is shown, by way of illustration, specific example embodiments. It is to be understood that changes can be made to such example embodiments without departing from the scope of the present disclosure.

As used herein, the terms “subject” or “individual” are equivalent to the term “patient” and refer to a mammalian organism, preferably a human being, who may be diseased with the condition (e.g., disease or disorder) of interest and who may benefit biologically, medically, or in quality of life from treatment for the condition.

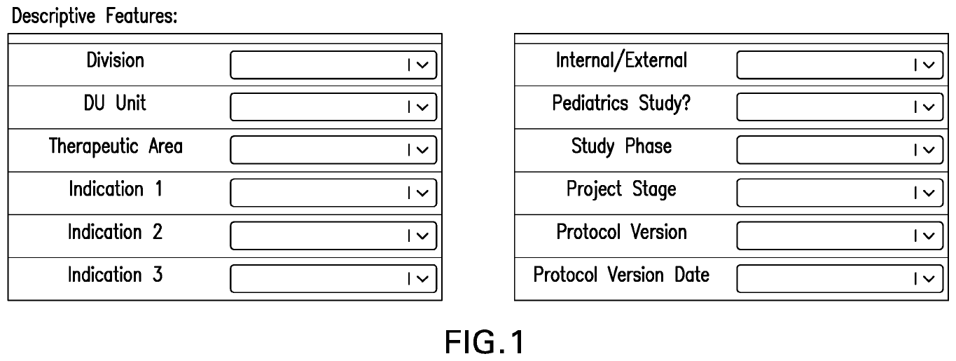

With initial reference to FIG. 1, a computing device implementing the method according to the present invention prompts an operator to input descriptive features in data entries correlated to a set-up of the a clinical trial study, in compliance with a clinical trial protocol. The data entry fields can be automatically generated by the system, provided a clinical trial protocol choice. The data may also be manually entered or adjusted by the operator.

The data for such entry fields can be automatically pulled from cooperating database software tools that facilitate the authoring and approval on clinical trial synopses and protocols, such as from Novartis' Collaborative Authoring Tool, or CAT; and/or from cooperating database software tools supporting the monitoring and management of the different phases of clinical trial studies, at an overall trial, country and site level, like Novartis's International Management Package for Administration of Clinical Trials, or IMPACT. Incidentally, Novartis' IMPACT provides information used to operationally track clinical trials including trial progress, site recruitment, and personnel trial participation (both site and internal personnel), the information being used for regulatory reporting as well as to support internal metrics, and investigator payments. In FIG. 1, the data for Therapeutic Area, Protocol Version and Protocol Version Date are pulled from CAT; whereas the data relating to Indications, Study Phase, Project Phase and Pediatrics Study are pulled from IMPACT. The operator has the ability to overwrite the automatic update if required or manually enter the information also.

The data to be collected in FIG. 1 preferably does not directly impact the complexity score and are considered descriptive data points only per clinical trial protocol.

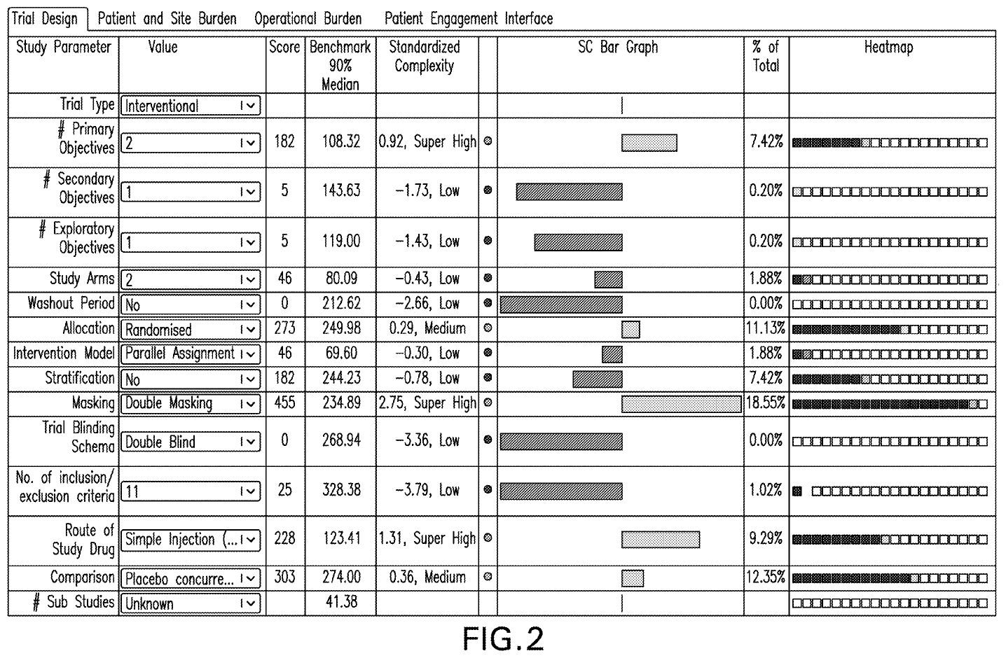

In general, data to be filled in the data entry fields shown in FIG. 1 and in following FIGS. 2, 3, 6 can be automatically populated from cooperating databases, following the choice of a clinical trial study protocol; and/or can be manually filled in by an operator.

FIG. 2 illustrates an example of parameters referring to objectives; design; methods; patient assessment procedures and patient data collection modes in a Trial Design pillar, or portion, of a clinical trial study. In this specific example, the Trial Design pillar comprises 14 parameters, namely: Trial type, as an informative only parameter; Primary Objectives; Secondary Objectives; Exploratory Objectives; Study Arms; Washout Period; Allocation; Intervention Model; Stratification; Masking; Trial Blinding Schema; Number of inclusion/exclusion criteria; Route of Study Drug; Comparison; Number of Sub Studies. By way of example, Objective parameters are drawn from CAT; whereas Allocation; Intervention Model; Trial Blinding Schema and Route of Study Drug are drawn from IMPACT. The parameter Stratification can capture whether, following randomization, the specific clinical trial study stratifies subjects based on characteristics such as age, gender, genotypes etc. The parameter comparison can take into account whether the study drug is compared at varying doses against placebo or active treatment (dose comparison); or instead whether the study drug is compared against active treatment and placebo (active treatment or placebo comparison); or instead just to the placebo.

With reference to FIG. 2, the method according to the present invention calculates the complexity score value for the Trial Design pillar of the exemplary clinical trial study, by averaging the scaled-namely, standardized-score values of each parameter comprised in the pillar based. The standardized score values for each of the parameters in the Trial Design pillar are given in the “standardized complexity” column. As above described in the general disclosure, these result from multiplying the score value of each listed parameter, as drawn from a score table, by an adjustment factor out of a Patient and Site Burden Part B Assessment, to produce an “x” adjusted score value for each parameter, prior to the standardization operation.

The graphic user interface is so conceived that a standardized complexity value (SD) more than 0.4 standard deviations above the benchmark is marked “high”; more than 0.8 standard deviations above the benchmark is marked “super high” while values with lower complexity are labelled “low deviation” (i.e., negative distance, less than or equal to 0), and values up to 0.4 standard deviations above the benchmark are labelled “medium”.

Moreover, also a benchmark-independent, non standardized parameter score is shown in heatmaps, where an operator can see how much each parameter contributes to the overall, non standardized complexity score. This additional feature is designed to show how much each adjusted, non-standardized score value is contributing the complexity across all (pillar and overall clinical trial study) parameters, providing the percentage distribution of each parameter against the total complexity score.

The Trial Design pillar complexity score in the example of FIG. 2 is given by averaging the standardized score values of all 14 parameters comprised in the pillar.

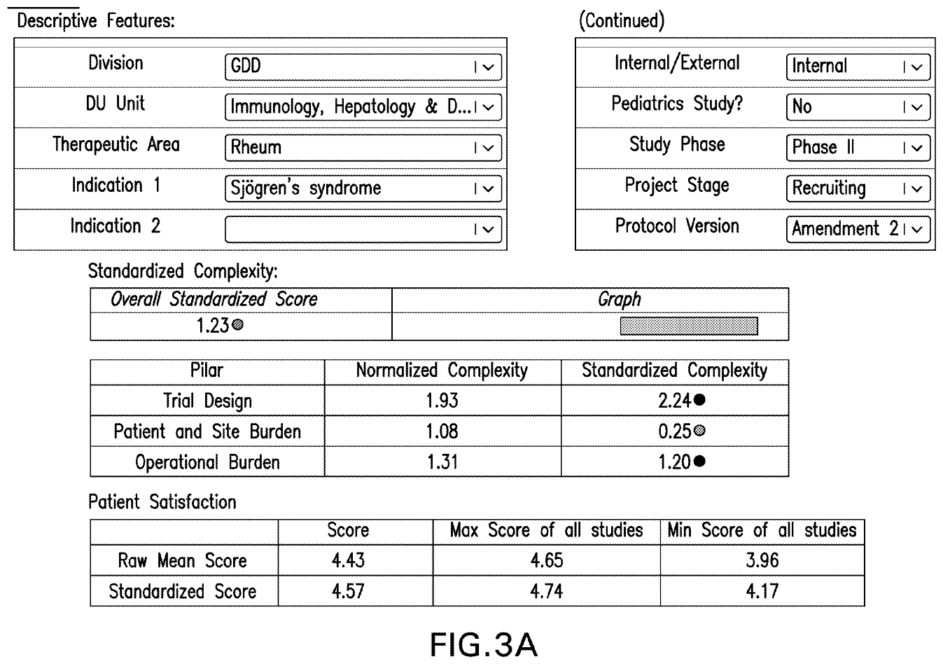

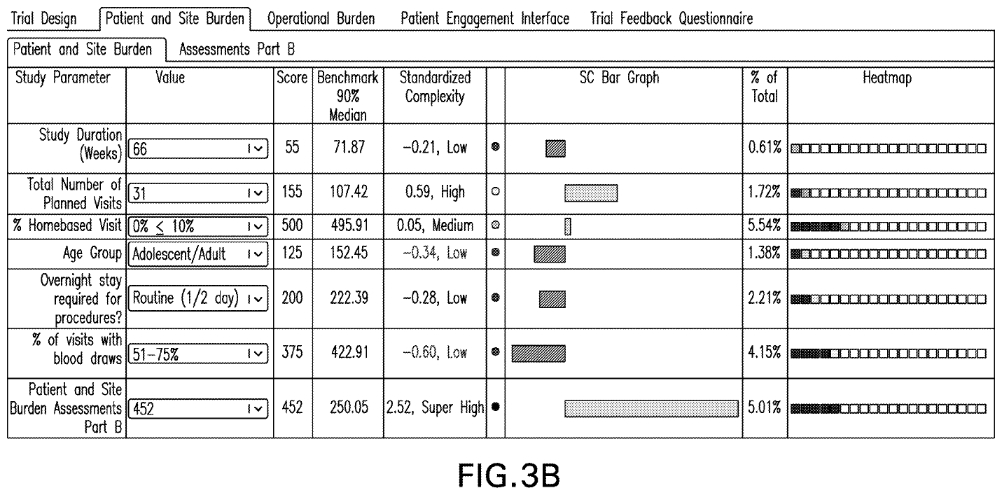

With reference to FIG. 3, an example of parameters is given referring to design; patient data; patient assessment procedures; and patient data collection modes in a Patient and Site Burden pillar, or portion, of a clinical trial study. In this specific example, the Patient and Site Burden pillar comprises 7 parameters, namely: Study Duration (in Weeks); Total Number of Planned Visits; % Homebased Visits; Age Group; Overnight stay for study procedures; % of visits with Blood Draws; and Patient and Site Burden Assessments Part B.

By way of example, the parameter Age Group can be pulled from IMPACT; whereas the parameters Total Number of Planned Visits and Patient and Site Burden Assessments Part B can be pulled from CAT. The operator has ability to enter data manually, should this be required.

Similarly to the pillar of FIG. 2, in the case of FIG. 3 the adjusted score values “x” for each parameter of the Patient and Site Burden pillar, prior to the standardization operation, are given in the “Score” column. The adjusting factor, or weight, applied to the parameters' score values in the specific case can be 452, being the Patient and Site Burden Assessments Part B score of 452. In some other embodiments, the adjusting factor, or weight, applied to the parameters' score values can be based on the average of the Part B for all studies, i.e. across all trials to-date, for instance to be divided by 100, rather than on the Patient and Site Burden Assessments Part B score for the current study. The standardized score values-with respect to the “Benchmark 90% Median”, are given in the “Standardized Complexity” column. As above explained, the “Benchmark 90% Median” values are calculated applying a 90% Trim Mean function to the benchmark database.

Analogously to the pillar shown in FIG. 2, the graphic user interface shows the SD score for each parameter in the Patient and Site Burden pillar. In the specific example of FIG. 3, the parameter “Patient and Site Burden Assessment Part B”, having more than 0.8 standard deviations above the benchmark, is marked super high”; the parameter “Total Number of Planned Visits”, having standardized complexity greater than 0.4 standard deviations above the benchmarks, in marked as “high”; the parameter “% Homebased Visit”, having complexity level values up to 0.4 standard deviations above the benchmark, is marked “medium”; whereas the parameters “Study Duration”, “Age Group”, “Overnight stay” and “% of visits with blood draws”, whose values have negative distance, less than or equal to 0, with respect to the benchmark, are marked “low”. Accordingly, the adjacent bar graph indicates complexity in relation to the benchmark median, bars to the left of the X axis indicating low complexity and bars to the right of the x-axis indicating high complexity.

The heatmaps show how much each parameter contributes to the overall, non standardized complexity score, independent from the benchmark. The parameters “% Homebased Visit” and “Patient and Site Burden Assessment Part B” appear as the most relevant in absolute terms.

In the example of FIG. 3, the Patient and Site Burden pillar is marked as having a 1.08 normalized complexity, as a result of a sum of adjusted complexity scores for all parameters, divided by the sum ‘Benchmark 90% median’ for the pillar. The same Patient and Site Burden pillar has pillar standardized complexity score, calculated as an average of all 7 parameters' standardized complexity scores for the pillar, of 0.25.

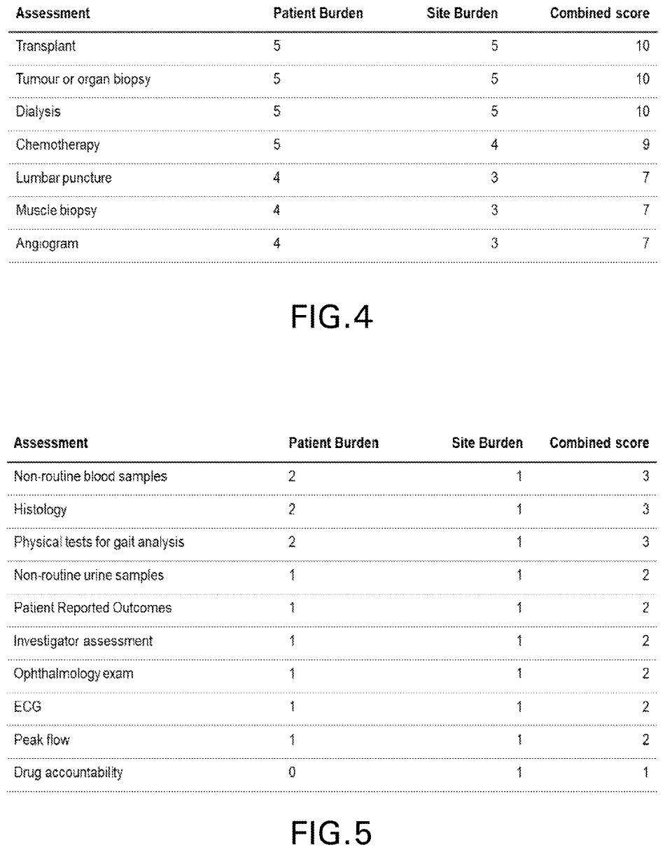

FIG. 4 provides an example of parameters for patient assessment procedures within the Patient and Site Burden pillar, which have high complexity score values, based on two components comprising a score value respectively for the Patient portion and for the Site portion. Transplant, tumor or organ biopsy and dialysis are procedures which are the most burdensome within the Patient and Site Burden pillar.

FIG. 5 provides an example of parameters for patient assessment procedures within the Patient and Site Burden pillar, which have low complexity score values, based on two components comprising a score value respectively for the Patient portion and for the Site portion. ECG, peak flow and drug accountability are examples of measurements and records which imply the least of complications within the Patient and Site Burden pillar.

Incidentally, a drug accountability record is a log of study drugs kept by an investigator running a clinical trial, listing notes about each drug, including the drug name, lot number, expiration date, the amount of drug received, used, returned, or thrown away, and the amount left. Drug Accountability Records help make sure that a clinical trial is done safely and correctly, in compliance with requirements by the U.S. Food and Drug Administration (FDA). Peak flow measurement is a quick test to measure air flowing out of the lungs.

Analogously to the examples given for the pillars Trial Design and Patient and Site Burden respectively in FIGS. 2 and 3, FIG. 6 illustrates an example of parameters referring to objectives; design; methods; patient assessment procedures and patient data collection modes in an Operational Burden pillar, or portion, of a clinical trial study. In this specific example, the Operational Burden pillar comprises 8 parameters, namely: Number of Countries; Number of Sites; % Expected Screen Fail Rates; Total Patients; Roll-over Study Design; Adjudication; Number of Interim Analysis; and Number of Vendors. By way of example, data on Number of Countries; Number of Sites; Total Patients and Number of Vendors can be drawn from IMPACT.

Also similarly to the outer pillars' examples introduced above, the method according to the present invention calculates the complexity score value for the Operational Burden pillar of the exemplary clinical trial study, by averaging the scaled-namely, standardized-score values of each parameter comprised in the pillar. The standardized score values for each of the parameters in the Operational Burden pillar are given in the “standardized complexity” column. As above described in the general disclosure, these result from multiplying the score value of each listed parameter, as drawn from a score table, by an adjustment factor out of a Patient and Site Burden Part B Assessment, to produce an “x” adjusted score value for each parameter, prior to the standardization operation.

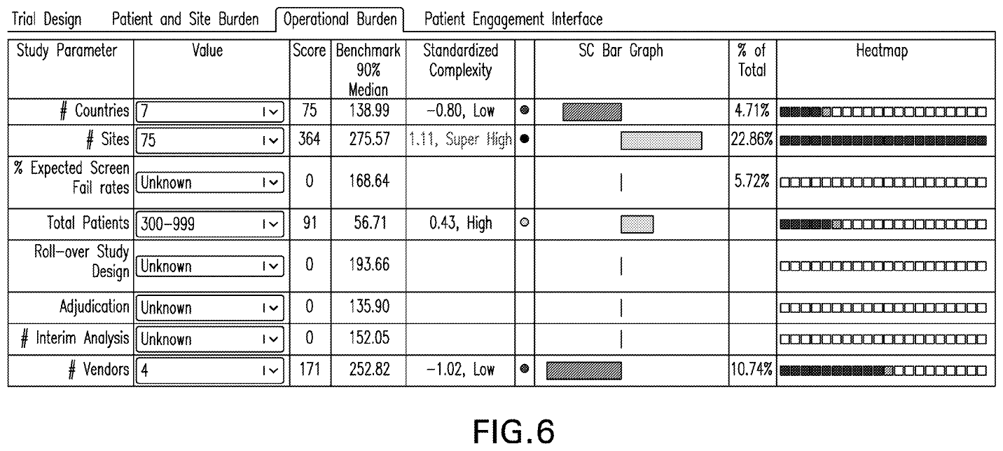

The Operational Burden pillar complexity score in the example of FIG. 6 is given by averaging the standardized score values of all 8 parameters comprised in the pillar.

In the specific example of FIG. 6, the parameter “Number of Sites”, having more than 0.8 standard deviations above the benchmark, is marked “super high”; the parameter “Total Patients”, having complexity level values more than 0.4 standard deviations above the benchmark is labelled “high”; while the parameters “Number of Countries” and “Number of Vendors” whose values have negative distance, less than or equal to 0, with respect to the benchmark, are labelled “low”. Accordingly, the adjacent bar graph indicates complexity in relation to the benchmark median, with bars to the left of the X axis indicating low complexity for “Number of Countries” and “Number of Vendors” and bars to the right of the X axis indicating high complexity for “Number of Sites” and “Total Patients”. The length of the bars is evidently proportional to the complexity level respectively assessed. In absolute terms as derivable from the heatmaps, irrespective of benchmarks and against the total complexity score, the parameters relating to the number of vendors and the number of sites drive the complexity for the Operational Burden pillar of FIG. 6.

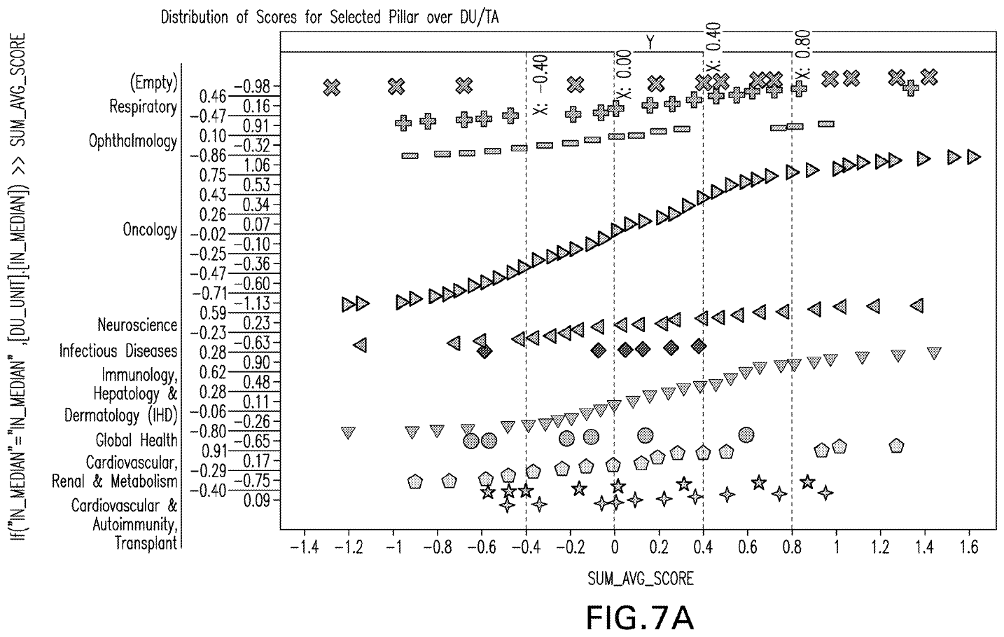

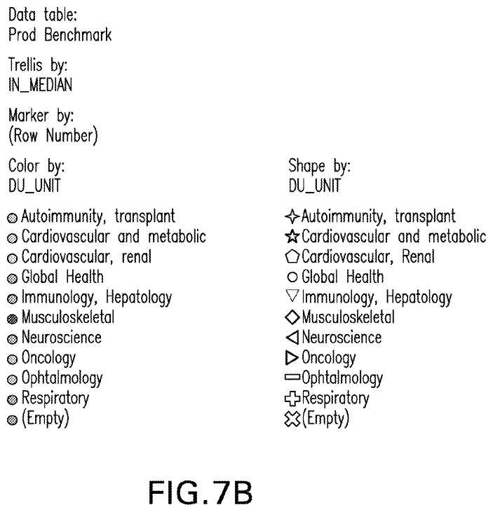

FIG. 7 shows an example of a graph created by the software of the present invention, for visualizing the overall standardized complexity scores of selectable clinical trial studies, in relation to other clinical trial studies comprised in the benchmark. Different therapeutic areas or developmental units (such as respiratory; ophthalmology; oncology; immunology, hepatology and dermatology; cardio renal and metabolics etc.) are labelled with various shapes as shown in the legend. Other graph views relating to the same clinical trial studies can be shown, wherein instead the values visualized can focus on the standardized complexity scores for each of the pillars, e.g. distinctly for each of the trial design, patient & site burden, operational burden pillars. Filtering tools can be used by an operator to filter results e.g. by each of the 29 parameters, or by Developmental Unit (DU) or by study phase.

By either clicking and dragging the mouse to “lasso-select” a group of data-points, or by selecting a single data point, out of the visualization of FIG. 7, the study codes of the corresponding clinical trial studies in the benchmark comprised in the selection can be displayed. Matching complexity scores, either partial for a filtered pillar or overall, can be highlighted across all possible alternative views, for ease of comparison and reference.

In general, the system can offer the possibility to filter complexity level results in the benchmark, by any of the descriptive features as listed in FIG. 1, e.g. by division, developmental unit, therapeutic are, indication or phase of study etc.

Thus, an operator can quickly get an idea, through the above interactive graphic user interface, of what portion of a clinical trial study is, for instance, dictating the complexity level compared to other studies in an updated benchmark, either within one same developmental unit or across all kinds of clinical trials; or of differences in how a given parameter drives the complexity in several distinct clinical trial studies.

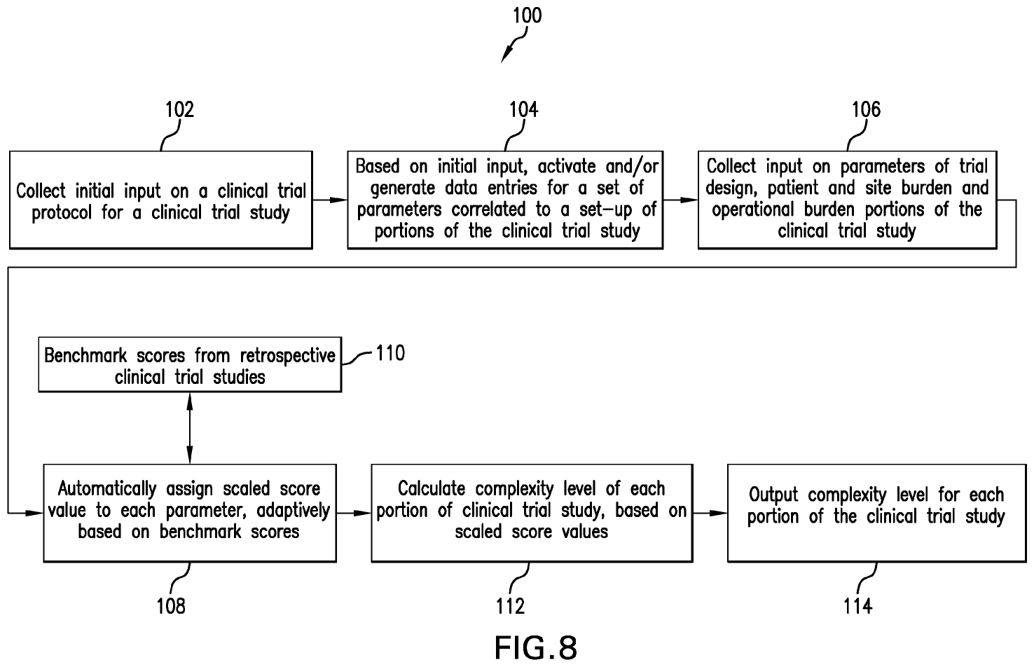

FIG. 8 is a process flowchart in diagrammatic form of exemplary embodiments of the present invention. In FIG. 8, the overall clinical trial protocol evaluation process is represented by the numeral 100. Within the process 100, block 102 represents an initial step of collecting input data. The input data is then used in block 104 to initiate parametric data entries. In block 106 data is collected on the parametric data entries, for example in respect of trial design and patient, site, and operational burdens of a clinical trial study. The information and data from block 106 is then input at block 108 into an appropriate processor of the system 100 which automatically and adaptively assigns scaled score values to each parameter. At this point benchmark scores from block 110 are additionally input into the system at block 108. Subsequently, a calculation step is performed at block 112, and an clinical trial complexity level is output at block 114.

In the embodiment of an overall process flow, and further referring to FIG. 8 a clinical trial design may start by defining a clinical trial protocol. Based on the defined protocol, the system activates and/or generates data entries for a set of corresponding parameters correlated to a set-up of a portion of the clinical trial study. In one example, fourteen weighted parameters which are entered into the system. Once the trial design is established, patient and site burden are evaluated. In one example, six weighted parameters may be evaluated with respect to site activities and patient activities. Next, an additional set of patient and site burdens are evaluated, in this instance related procedures required from patients and sites. Both the activities burdens and procedures burdens are mathematically manipulated to arrive at an overall burden score. An operational burden is calculated by defining operational parameters of the study including for example number of countries, number of sites, number vendors and definable risks. Finally the system computes, as a final outcome, a complexity assessment including an identification of factors that drive complexity. The latter is important because the identification of complexity factors can enable the clinical trial designers to mitigate, eliminate, or adjust such complexity factors for any given trial. In some embodiments, such complexity factors can be manipulated through sliders or toggles, enabling the clinical trial designer to see a real-time display of overall complexity assessment.

This description and the accompanying drawings that illustrate aspects and embodiments of the present invention should not be taken as limiting the claims defining the protected invention. In other words, while the invention has been illustrated and described in detail in the drawings and foregoing description, such illustration and description are to be considered illustrative or exemplary and not restrictive. Various mechanical, compositional, structural, electrical, and operational changes may be made without departing from the spirit and scope of this description and the claims. In some instances, well-known circuits, structures and techniques have not been shown in detail in order not to obscure the invention. Thus, it will be understood that changes and modifications may be made by those of ordinary skill within the scope and spirit of the following claims. In particular, the present invention covers further embodiments with any combination of features from different embodiments described above and below.

The disclosure also covers all further features shown in the Figs, individually although they may not have been described in the afore or following description. Also, single alternatives of the embodiments described in the figures and the description and single alternatives of features thereof can be disclaimed from the subject matter of the invention or from disclosed subject matter. The disclosure comprises subject matter consisting of the features defined in the claims or the exemplary embodiments as well as subject matter comprising said features.

Furthermore, in the claims the word “comprising” does not exclude other elements or steps, and the indefinite article “a” or “an” does not exclude a plurality. A single unit or step may fulfil the functions of several features recited in the claims. The mere fact that certain measures are recited in mutually different dependent claims does not indicate that a combination of these measures cannot be used to advantage. The terms “essentially”, “about”, “approximately” and the like in connection with an attribute or a value particularly also define exactly the attribute or exactly the value, respectively. The term “about” in the context of a given numerate value or range refers to a value or range that is, e.g., within 20%, within 10%, within 5%, or within 2% of the given value or range. Components described as coupled or connected may be electrically or mechanically directly coupled, or they may be indirectly coupled via one or more intermediate components. Any reference signs in the claims should not be construed as limiting the scope.

1. A computer-implemented method for assessing and determining a complexity level of at least a portion, or pillar, of a clinical trial study, comprising the steps of:
at a computing device including at least a processor; a memory and an input and/or output elements,
by the input element, selecting and/or defining a clinical trial protocol and, based on the selection and/or definition thereof, activating and/or generating data entries for a set of corresponding parameters correlated to a set-up of the portion of the clinical trial study;
wherein the parameters refer to objectives; and/or design; and/or methods; and/or
patient assessment procedures and/or patient data collection schedules of the clinical trial study;
by the processor,
automatically assigning a score value to each parameter in the set of parameters,
applying statistical rules to the score value of each parameter to obtain a scaled score value; and
calculating a complexity level for the portion of clinical trial study, based on the scaled score values of each parameter in the set of parameters;
wherein the score value of each parameter is adaptively based on benchmark score values of retrospective/previously assessed clinical trial studies for the same parameter.

2. The method of claim 1, wherein the scaled score value of each parameter is the standardized score value (SV) for each parameter, calculated according to the following:
SV=(x−mean)/standard deviation
wherein
“x” is an adjusted score value for each parameter, derived from a predefined scoring table reflecting the relative importance of the score value of the parameters;
“mean” is the mean of each score value, based on the benchmark score values of the retrospective/previously assessed clinical trial studies for the same parameter; and
“standard deviation” is a value calculated as the square root of variance, by determining each parameter's score value deviation relative to the mean, measuring the amount of variation, or dispersion, of a distribution of the score values of a parameter in the set.

3. The method of one of claim 1 or 2, wherein, when selecting and/or defining a clinical trial protocol, the computing device automatically presents an initial user interface to an operator, comprising a preliminary multiplicity of clinical trial protocol data entries activated and/or generated based on an initial choice by an operator of at least one of:
a category of therapeutic area; a developmental unit; and
subsequently the computing device presents to the operator clinical trial protocol data entries adaptively adjusted, based on a probability value that the set of corresponding parameters drives the complexity level of the portion of the clinical trial study.

4. The method of one of claims 1 to 3, wherein the clinical trial study comprises at least one, or a combination, of the following portions, or pillars:
a Trial Design pillar; a Patient & Site Burden pillar; an Operational Burden pillar;
wherein:
for each portion, or pillar, of the clinical trial study, a pillar complexity score value is calculated by averaging the scaled score value of each parameter comprised in each portion, or pillar.

5. The method of one of claims 1 to 4, wherein an overall complexity level score value for the clinical trial study is calculated by averaging the scaled score values determined for each of the portions, or pillars.

6. The method of one of claims 1 to 5, wherein for each parameter, a score value from a scoring table is applied, the median for each scored parameter being set at 100.

7. The method of one of claims 2 to 6, wherein for each parameter, the mean of each score value equals to the 90% Trim Mean, that is the mean of the middle 90% benchmark score values for the same parameter as derived from the retrospective/previously assessed clinical trial studies.

8. The method of one of claims 4 to 7, wherein the Trial Design pillar comprises 14 parameters; the Patient & Site Burden pillar comprises 7 parameters; and the Operational Burden pillar comprises 8 parameters; the clinical trial study comprising overall 29 parameters.

9. The method of one of claims 1 to 8, comprising an adjustment factor, or weight, for the score value of each parameter, to obtain an adjusted score value, applicable prior to the calculation of the standardized score value (SV); the adjustment factor, or weight being configured to prevent that a score value of any parameter dominates over the score value of other parameters.

10. The method of claim 9 when depending from claim 4, wherein the adjustment factor, or weight, is calculated based on the Patient & Site Burden pillar score value, to prevent that the Patient & Site Burden pillar score value dominates over score values of other parameters.

11. The method of claim 10, wherein the adjustment factor, or weight, is calculated as the average complexity score value of the Patient & Site Burden pillar, calculated across all clinical trial studies comprised in the benchmark of retrospective/previously assessed clinical trial studies, divided by an estimated median of each scored parameter.

12. The method of claim 11, wherein each parameter comprised in the Patient & Site Burden pillar is assigned a score value from a distinct, pre-defined scoring table; each score value respectively for the Patient portion and for the Site portion of the Patient & Site Burden pillar being multiplied by the number of occurrences during the clinical trial study and subsequently summed.

13. The method of one of claims 1 to 12, wherein the computing device comprises a graphical user interface configured to visually convey to a user, or operator, a measure of the impact on the complexity level of each portion, or pillar, and/or of each parameter comprised in the clinical trial study, as a result of a current selection and/or definition of a clinical trial protocol.

14. The method of claim 13, wherein the graphical user interface is interactive in that it changes in real-time, based on modified selection and/or definition of a clinical trial protocol by a user, or operator.

15. The method of claim 13 or 14, wherein the graphical user interface allows to visualize the complexity level of the clinical trial study (as an overall standardized score value); or of a portion, or pillar, thereof, in relation to other clinical trial studies comprised in the benchmark of retrospective/previously assessed clinical trial studies.

16. The method of claim 15, wherein the graphical user interface allows to visualize the complexity level of the clinical trial study, either within clinical trial studies belonging to a specific developmental unit; or across all clinical trial studies; or filtering by clinical trial study phase.

17. The method of any one of claims 1 to 16, wherein the benchmark score values of the retrospective/previously assessed clinical trial studies are updated and stored on the memory, prior to applying statistical rules to the score value of each parameter for assessing and determining the complexity level of the portion, or pillar, of the clinical trial study.

18. A system comprising:
a computing device including:
one or more processors;
one or more input and/or output elements;
memory; and
one or more programs stored in the memory, the one or more programs including instructions for:
by the input element, selecting and/or defining a clinical trial protocol and, based on the selection and/or definition thereof, activating and/or generating data entries for a set of corresponding parameters correlated to the set-up of the portion of the clinical trial study;
wherein the parameters refer to objectives; and/or design; and/or methods; and/or patient assessment procedures and/or patient data collection schedules of the clinical trial study;
by the processor,
automatically assigning a score value to each parameter in the set of parameters, applying statistical rules to the score value of each parameter to obtain a scaled score value; and
calculating a complexity level for the portion of clinical trial study, based on the scaled score values of each parameter in the set of parameters;
wherein the score value of each parameter is adaptively based on benchmark score values of retrospective/previously assessed clinical trial studies for the same parameter.

19. A non-transitory computer-readable storage medium storing one or more programs configured to be executed by one or more processors of an electronic device with one or more input and/or output elements, the one or more programs including instructions for:
by the input element, selecting and/or defining a clinical trial protocol and, based on the selection and/or definition thereof, activating and/or generating data entries for a set of corresponding parameters correlated to the set-up of the portion of the clinical trial study;
wherein the parameters refer to objectives; and/or design; and/or methods; and/or patient assessment procedures and/or patient data collection schedules of the clinical trial study;
by the processor,
automatically assigning a score value to each parameter in the set of parameters,
applying statistical rules to the score value of each parameter to obtain a scaled score value; and
calculating a complexity level for the portion of clinical trial study, based on the scaled score values of each parameter in the set of parameters;
wherein the score value of each parameter is adaptively based on benchmark score values of retrospective/previously assessed clinical trial studies for the same parameter.
